(19) United States
(12) Patent Application Publication
(10) Pub. No.: US 2021/0052330 A1
(43) Pub. Date: Feb. 25, 2021

(54) INTEGRATED MEDICAL IMAGING SYSTEM FOR TRACKING OF MICRO-NANO SCALE OBJECTS

(71) Applicants: Alex KISELYOV, San Diego, CA (US); BIONAUT LABS LTD., Herzliya (IL)

(72) Inventors: Alex KISELYOV, San Diego, CA (US); Michael SHPIGELMACHER, Los Angeles, CA (US); Dina SHENKAR, Baltimore, MD (US); Eran OREN, Tel Aviv (IL); Michael KARDOSH, Kiryat Ono (IL); Eldad ELNEKAVE, Tel Aviv (IL); Edward GAO, Alhambra, CA (US); Suehyun CHO, Los Angeles, CA (US); John CAPUTO, Los Angeles, CA (US); Dennis SEELY, Temecula, CA (US)

(73) Assignee: BIONAUT LABS LTD., Herzliya (IL)

(21) Appl. No.: 17/052,118

(22) PCT Filed: May 2, 2019
(86) PCT No.: PCT/US2019/030390

§ 371 Date: Oct. 30, 2020

Related U.S. Application Data
(60) Provisional application No. 62/666,517, filed on May 3, 2018.
(60) Provisional application No. 62/754,834, filed on Nov. 2, 2018.

(51) Int. Cl. A61B 34/20; A61B 8/08; A61B 8/00; A61B 6/03; A61B 5/05; A61B 6/00; A61B 5/00

(52) U.S. Cl. CPC A61B 34/20; A61B 8/0833; A61B 8/461; A61B 6/032; A61B 8/5269; A61B 2034/2055; A61B 5/0515; A61B 6/463; A61B 5/0071; A61B 5/0077; A61B 2034/2063; A61B 8/488

(57) ABSTRACT

Apparatus and methods for imaging and tracking of nano- and micro-scale objects with acceptable latency for relevant medical procedures, such as delivery of therapeutic payload or minimally invasive surgery are disclosed, including the capability to superimpose accurate anatomical data over a tracking image. Software applications are provided for data logging via a remote-control station; and software interface with remote motion control mechanism, controlling the motion of internal device.

### BACKGROUND OF THE INVENTION

Remote-control of medical devices moving inside the human body (internal devices) can be useful for a variety of purposes, including delivery of therapeutic payloads, diagnostics or surgical procedures. Such internal devices may include micro or nano scale robots, medical tools, “smart pills”, etc. Such devices may be able to move in the body either through self-propulsion or an external propulsion mechanism. Accurate location and tracking of such internal devices may be necessary to ensure their proper functioning at the right anatomical location.

However, designing a practical medical imaging system, which can accurately track the internal devices, is a technical challenge. Specifically, the critical parameters which need to be balanced include the frequency of sampling, latency, maximal depth of imaging inside tissue, spatial resolution, imaging system cost, medical safety and ability for anatomic feature overlay. Accordingly, there is a long felt need for an integrated medical imaging system, allowing the following types of functionality:
i. Imaging and tracking of nano-micro scale objects with acceptable latency for relevant medical procedures, such as delivery of therapeutic payload or minimally invasive surgery;
ii. Overlay (superimposing) of accurate anatomical data upon tracking image; iii. Adherence to medical safety regulations, including materials, therapeutic and/or diagnostic payload, radiation, temperature, ultrasound frequency;
iv. Data logging via a remote-control station; and
v. Software interface with remote motion control mechanism, controlling the motion of internal device.

### SUMMARY OF THE INVENTION

According to some embodiments, an integrated medical imaging system is provided, allowing the following five types of functionality:
i. Imaging and tracking of nano-micro scale objects with acceptable latency for relevant medical procedures, such as delivery of therapeutic payload or minimally invasive surgery;
ii. Overlay (superimposing) of accurate anatomical data upon tracking image;
iii. Adherence to medical safety regulations, including materials, therapeutic and/or diagnostic payload, radiation, temperature, ultrasound frequency;
iv. Data logging via a remote-control station; and
v. Software interface with remote motion control mechanism, controlling the motion of internal device.

According to some embodiments, an imaging system is provided configured for tracking nano- or micro-particles, the system comprising:
an ultrasound imager having plurality of ultrasound sensors driven by at least one ultrasound transducer signal, the imager is configured to sample at a sampling rate in the kHz-MHz range;
a plurality of particles having an image enhancement feature facilitating detection in a patient or an in-vivo environment, the particles having a size in a micrometer or nanometer range; and
a display configured to display the particles in the patient or in-vivo environment via the ultrasound imager.

According to some embodiments, the imaging system further comprising a low voltage CAT scan (CT) technology configured to display the particles in the patient or in-vivo.

According to some embodiments, the ultrasound imager is operative at a processing delay of one second or more.

According to some embodiments, the ultrasound imager is operative in accordance with a standard operating or optimized procedure providing multi-organ resolution of up to 50 microns.

According to some embodiments, the ultrasound imager is configured to process feedback signals through a specialized standard or custom algorithm so as to enhance signal-to-noise ratio (SNR).

According to some embodiments, the image enhancement feature is implemented as a coating containing iodine.

According to some embodiments, the image enhancement feature is implemented as a surface irregularity.

According to some embodiments, the image enhancement feature is implemented as a result of particle dynamics or specific motion frequency, as exemplified by Doppler effect.

According to some embodiments, the low voltage CT technology is operative at 50-300 kVolt.

According to some embodiments, the imaging system further comprising a magnetic imaging system configured to track the particles.

According to some embodiments, the imaging system further comprising a propulsion system configured to advance the particles through the patient or in-vivo environment via a series of magnetic propulsions.

According to some embodiments, the magnetic imaging system is configured to capture position images of the particles in the patient or in-vivo environment in between the magnetic propulsions.

According to some embodiments, the image enhancement feature is implemented as a load of a superparamagnetic iron oxide nanoparticles (SPION) and/or mesoporous silica nanoparticles (MSN).

According to some embodiments, a magnetic imaging system is provided configured for tracking conveyable, therapeutic nano- or micro-particles, the system comprising:
a magnetic imager configured to track the particles in a patient or an in-vivo environment;
a plurality of conveyable, therapeutic nano- or micro-particles loaded with superparamagnetic iron oxide nanoparticles (SPION) or mesoporous silica nanoparticles (MSN);
a display configured to display the particles in the patient or in-vivo environment, via the magnetic imager.

According to some embodiments, the magnetic imaging system further compromising a low voltage CAT scan (CT) technology configured to display the particles in the patient or in-vivo environment.

According to some embodiments, the low voltage CT technology is operative at 80 kVolt.

According to some embodiments, the magnetic imaging system further comprising a propulsion system configured to advance the particles through the patient or in-vivo environment through a series of magnetic propulsions.

According to some embodiments, the magnetic imager is configured to capture position images of the in the patient or in-vivo environment in between the magnetic propulsions.

According to some embodiments, the magnetic imaging system further comprising an ultrasound imager having a plurality of ultrasound sensors driven by a single ultrasound transducer signal.

According to some embodiments, the conveyable, therapeutic particles have an iodine coating.

According to some embodiments, a method is provided for tracking conveyable, therapeutic nano- or micro-particles in a patient or an in-vivo environment; the method comprising:
sampling the patient or the in-vivo environment at an ultrasound sampling frequency in kHz-MHz range with intermittent gaps of one or more seconds between subsequent ultrasound applications;
processing sample feedback of the conveyable, therapeutic particles in the patient or the in-vivo environment with a protocol providing multi-organ resolution up to 50 microns;

and
displaying the objects on a display.

According to some embodiments, the method further comprising: employing low voltage CAT scan (CT) technology, and displaying the CT scanned objects on a display.

According to some embodiments, the low voltage CT technology is operative at 80 kVolt.

According to some embodiments, the method further comprising propelling the conveyable, therapeutic particles in the patient or in-vivo environment through magnetic propulsions.

According to some embodiments, the method further comprising magnetically imaging the conveyable, therapeutic particles in the patient or in-vivo environment in between the magnetic propulsions.

According to some embodiments, an imaging system is provided configured for tracking nano- or micro-particles, the system comprising:
nano- or micro-particles having embedded rare earth ion-doped phosphors;
an upconversion energy source configured to provide energy sufficient to upconvert photons of the ion-doped phosphors to a visible range;
a detector having plurality of sensors configured to detect luminescence from the nano- or micro-particles; and
a display configured to display the particles in the patient or in-vivo environment based on the detected luminescence.

According to some embodiments, the detector is a system comprising a Complementary Metal Oxide Semiconductor (CMOS) detector and a shortpass filter positioned between the luminescence in the in-vivo environment and the CMOS detector; and wherein the upconversion energy source is a laser configured to provide excitation energy at a wavelength of 800.

According to some embodiments, the rare earth ion-doped phosphors comprise Yb³⁺ and/or Er³⁺ doped NaYF₄ crystals.

According to some embodiments, a method is provided for tracking a nano- or microparticle in an in-vivo environment, the method comprising:
coating at least one metallic nano- or microparticle with rare earth ion doped upconversion phosphors;
implanting the nano or microparticle coated with rare earth ion doped upconversion phosphors in said in-vivo environment;
exciting the upconversion phosphors to produce luminescence;
imaging the luminescence with a camera;
detecting the position of the nano- or microparticles in-vivo.

According to some embodiments, exciting the upconversion phosphors is done with a laser configured to provide excitation energy in a range of about 800 nm to about 980 nm, wherein said camera is a complementary metal oxide semiconductor (CMOS) detector, and said luminescence is visible red and/or green light.

According to some embodiments, the method further including moving the nano- or micro-particle in the in-vivo environment with a magnetic field.

According to some embodiments, a method is provided for making nano- or micro-particles configured to be tracked in an in-vivo environment, the method comprising:
providing a compression spring;
clipping an end of the compression spring to form a sharp end of the compression spring;
axially aligning a magnet with the compression spring;
positioning rare earth ions onto the magnet; and
adhering the magnet to the compression spring.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts a representative helical topology of a particle, according to some embodiments of the invention;

FIG. 2 depicts internal features in the microparticle that may enhance imaging, including but not limited to micro-nanocavities, micro-nanorattles, micro-nanoinclusions and other micro-nanoirregularities, according to some embodiments of the invention;

FIG. 3 depicts microbot particle insertion in the liver (right medial lobe) of anesthetized rat using plastic forceps, according to some embodiments of the invention;

FIG. 4 schematically depicts a magnet and ultrasound imaging setup, according to some embodiments of the invention;

FIGS. 5A and 5B schematically depict front and top views (respectively) of the magnet and ultrasound imaging setup, according to some embodiments of the invention;

FIG. 6A depicts an ultrasound image of a rat with microbot shown in a green box, according to some embodiments of the invention;

FIG. 6B depicts an ultrasound image of a spring-magnet based particle (spring-cylindrical magnet combo of approximately 3 millimeter (mm) length (L)×1 mm diameter (D)) as visualized with the 18 MHz probe, according to some embodiments of the invention;

FIG. 7A depicts a schematic front view of an upconversion phosphor embedded microbot; the tip of the microbot shows flat and sharp, a chisel-type edge, according to some embodiments of the invention;

FIGS. 7B and 7C depict side views of the microbot; the microbot having a metallic cylinder in the center is nickel-plated neodymium (N52) magnet (0.5 mm×1 mm) and the translucent layer surrounding the magnet is sodium yttrium fluoride micro-particles doped with ytterbium and erbium ions encased in cyanoacrylate adhesive, according to some embodiments of the invention;

FIGS. 8A, 8B and 8C depict fabrication of a microbot coated with UCPs, according to some embodiments of the invention;

FIG. 8D depicts a basic schematic of an upconversion imaging setup; a 980 nm laser source excites the specimen with upconversion phosphor (UCP); upon excitation of the 980 nm source, the specimen upconverts incident near-infrared photons into visible photons; both the scattered illumination and upconverted visible signals get collected a collection optics and a CMOS detector, according to some embodiments of the invention;

FIG. 8E depicts microbot illuminated with 980 nanometer (nm) laser showing a clear green luminescence, according to some embodiments of the invention;

FIG. 9A depicts an image of a UCP-embedded microbot underneath 5 mm thick sample of a pork with conventional imaging, according to some embodiments of the invention;

FIG. 9B depicts a 980 nm laser illumination with a faint, green upconverted luminescence from the UCPs through 5 mm sample of a pork, according to some embodiments of the invention;

FIG. 9C depicts bot when it is removed from underneath 5 mm sample of the pork and under same 980 nm laser irradiation; No green luminescence is visible, demonstrating that the green signal is coming from UCPs and not the laser, according to some embodiments of the invention;

FIG. 9D depicts a 980 nm laser illumination with a faint, orange upconverted luminescence from the UCPs through a liver of a turkey, according to some embodiments of the invention; and

FIG. 10 depicts a setup used for ex-vivo illumination, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The provided description contains several embodiments to design an imaging system with the above mention five functionalities, some of which include standalone or hybrid external devices, relying on ultrasound (“US”), computerized tomography (“CT” or “CAT” scan), X-ray, magnetic, optical, and other mechanisms. The imaging system described herein includes both hardware components (such as the imaging sensors, communication devices and auxiliary hardware equipment), software components (the imaging and control algorithm), and may rely on specific properties of the internal devices to enhance imaging capabilities.

### Ultrasound-Based Imaging System for Moving Particles:

In one embodiment, the imaging system relies on one or more ultrasound sensors to track moving particles in the patient body, in-vivo or in-vitro/ex-vivo. As used herein, “particles” includes nano-scale and micro-scale particles also sometimes referred to herein as a nano-bots and micro-bots, respectively.

In one embodiment, the imaging technique is configured for diagnostic and/or therapeutic purposes, and is provided by high-frequency ultrasound in the range of 0.25-50 MHz.

In this embodiment, the particles are designed to propel through diverse viscoelastic media, tissues and organs ex-vivo, in-vivo and in patients.

The aforementioned therapeutic particles are designed to be propelled via external stimuli including but not limited to electro-magnetic, acoustic, optical, thermal energy sources or a combination of thereof.

In one embodiment the particles (microparticles, micropropellers, microbots) may be configured of 50 nm-2,000 micro-meter (μm) in diameter, and 1 μm-5,000 μm in length.

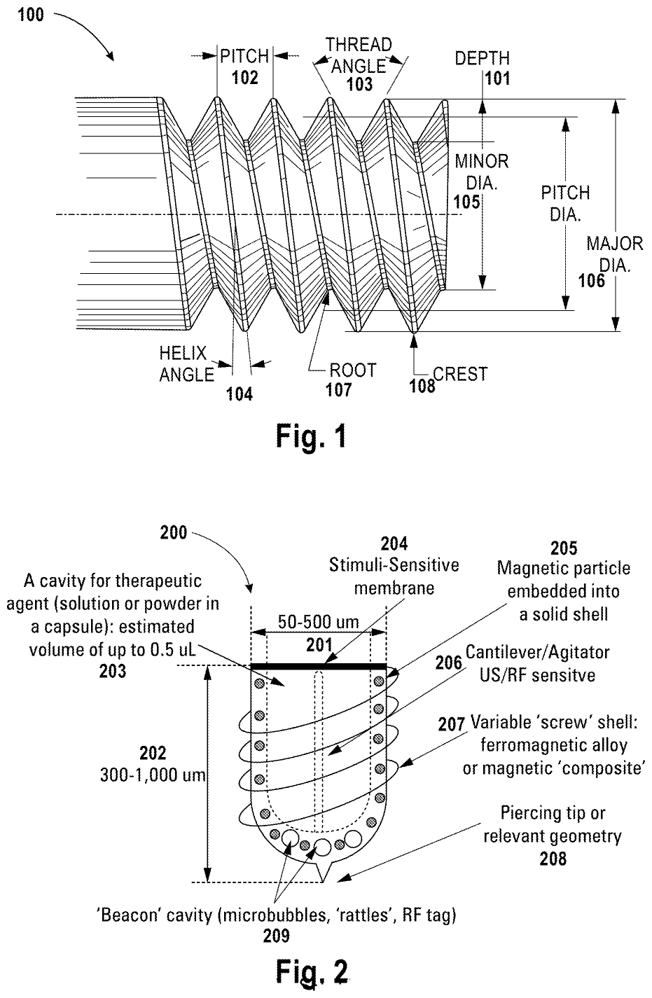

In one embodiment and as demonstrated in FIG. 1, the particles may feature a helical topology (referred to as propeller, drill, screw, etc. with specific depth (101), helical pitch (102), thread angle (103), helix angle (104), minor (105) and major (106) diameters, root (107) and crest (108) topology.

The aforementioned therapeutic particles are designed to facilitate imaging using diverse techniques including but not limited to magnetic, X-Ray, ultrasound, acoustic, radiofrequency, optical methods or a combination of thereof;

In one embodiment, therapeutic particles comprise magnetic, ferromagnetic, paramagnetic components or a combination of thereof, such as described in “Magnetic-Based Closed-Loop Control of Paramagnetic Microparticles using Ultrasound Feedback”, Islam S. M. Khalil, Pedro Ferreira, Ricardo Eleuterio, Chris L. de Korte, and Sarthak Misra, 2014 IEEE International Conference on Robotics & Automation (ICRA), Hong Kong Convention and Exhibition Center, May 31-Jun. 7, 2014, Hong Kong, China. Publications referenced herein are incorporated by reference. Reference to a publication, whether patent or non-patent is not an admission of the “prior art” status of such publication.

In one embodiment, therapeutic particles may include MEMS components, such as micro-cantilevers, membranes, etc., which can be used to enhance imaging.

In one embodiment, particles may be silica-based, as exemplified in Yang Zhou, et. al. “Construction of Silica-Based Micro/Nanoplatforms for Ultrasound Theranostic Biomedicine,” Adv. Healthcare Mater. 2017, DOI: 10.1002/adhm.201700646.

In one embodiment, therapeutic particles exhibit specific features created or added to enhance respective imaging via aforementioned methods or their combination.

In one embodiment, therapeutic particles exhibit specific features created or added to enhance respective imaging in specific media including but not limited to ex-vivo viscoelastic matrices, ex-vivo and in-vivo tissues, tissue combinations, organs or in patients.

In one embodiment, therapeutic particles exhibit specific features created or added to reduce background or artefactual features of specific media including but not limited to ex-vivo viscoelastic matrices, ex-vivo and in-vivo tissues, tissue combinations, organs or in patients.

In one embodiment, therapeutic particles may display a specific geometry or surface topology to facilitate imaging.

In one embodiment, therapeutic particles may exhibit a specific surface coating and/or multi-layered composition to facilitate image enhancement. Examples of such particle design can be found in: Kun Zhang, et. al. “Double-scattering/reflection in a Single Nanoparticle for Intensified Ultrasound Imaging,” Sci. Rep. 2015, 10.1038/srep08766; Jun Chen, et. al., “Theranostic Multilayer Capsules for Ultrasound Imaging and Guided Drug Delivery,” ACS Nano 2017, 11, 3135-3146: Dennis Manuel Vriezema, et. al. “Coating for Improving the Ultrasound Visibility”; and US 2014/0207000 A1.

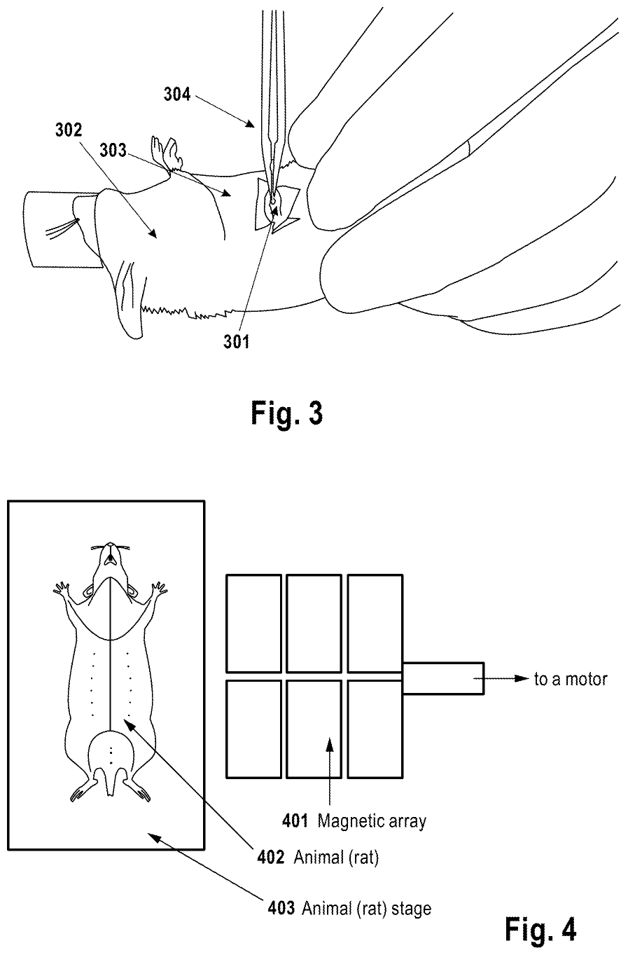

In one embodiment, therapeutic particles may exhibit specific internal compartments designed to enhance imaging as exemplified but not limited to micro-nanocavities, micro-nanorattles, micro-nanoinclusions or other micro-nanoirregularities, as exemplified in FIG. 2. In a related embodiment, a particle (200) can comprise at least one of the group consisting of:
a diameter (201) within the range: 50-500 μm
a length (202) within the range: 300-1000 μm;
a cavity (203) for therapeutic agent (solution and/or powder in a capsule) with an estimated volume of up to 0.5 micro-liter (μL);
a stimulus sensitive membrane (204);
magnetic particles embedded inti a solid shell (205);
cantilever/agitator (206) US/CT sensitive;
a variable screw shell (207) comprising ferromagnetic alloy and/or magnetic composite;
a piercing tip (208) or any relevant geometry; and
a beacon cavity (209) for microbubbles, rattles or RF tags.

In one embodiment, therapeutic particles may exhibit specific image enhancing inclusions, cavities, containers that release agents or modalities that enhance imaging as exemplified but not limited to contrasting agents or microbubbles. Examples of such particles are described in Shu-Guang Zheng, et. al. “Nano/microparticles and ultrasound contrast agents,” World J Radiol 2013 Dec. 28; 5(12): 468-471.

In one embodiment, therapeutic particles may exhibit specific image enhancing inclusions, cavities, containers that release agents or modalities that reduce background signal thus enhancing therapeutic particle signal.

In one embodiment, therapeutic particles may feature particular chemical and/or biochemical molecules, as exemplified by but not limited to Janus alloys, (micro)electrodes, Pd/Pd-alloys, specific redox or metabolic enzymes that facilitate local production of externally traceable substances that include gases and/or specific metabolites.

In one embodiment, therapeutic particles may exhibit specific dynamics and/or motion behavior to facilitate imaging as exemplified by off-gradient axis rocking, rotation, vibration, etc. In one embodiment, therapeutic particles may exhibit specific detectable movement dynamics as exemplified by the Doppler effect. Specifically, the aforementioned particle could be identified and localized using the Doppler signal caused its movement, including the frequency of rotation; alternatively, the Doppler image of the magnetic field could be recorded in order to filter out and identify the particle movement.

In one embodiment, the ultrasound system may use multiple ultrasound sensors in parallel, where the averaging of images reduces the noise under an independent noise assumption. The aforementioned ultrasound system exhibits at least one of the following specifications:
Reliable and reproducible calibration protocol suitable for multi-organ resolution of up to 50 μm or better as measured in any dimension;
Latency of up to 1 second or better to facilitate longitudinally resolved feedback during in vitro, in-vivo or clinical procedures;
Sampling frequency in the kHz range or more allowing tracking of objects moving at lateral speeds up to 60 centimeter (cm) per hour;
Reliable and reproducible resolution of 50 μm or better to facilitate unequivocal identification and location of particles featuring size 50 μm or larger in any dimension of measurement;
Reliable and reproducible detection of aforementioned particle in any practical observation plane; and
Reliable, reproducible detection of aforementioned particles at the detection depth of 30 cm or less.

### Overlay of Anatomical Features and Predefined 3D Locations in Ultrasound Imaging Space

In one embodiment, the aforementioned therapeutic particles may be delivered or propelled to a specific anatomical target as determined via diverse imaging techniques above, either pre-recorded or imaged in real time. For instance, it is possible to pre-scan the patient using X-Ray, CT, or another imaging modality, utilizing fiducial markers clearly visible on the pre-recorded anatomical image and on the ultrasound image. The location of the fiducial markers allows accurate superimposing of the anatomical features on the image of the tracked particle embedded in tissue. Note that this functionality is feasible for real time capturing of anatomical features in parallel to ultrasound imaging, as well as for pre-recording.

In one embodiment, the aforementioned therapeutic particles may be delivered or propelled to a specific predetermined locus as determined via pre-administered agents. The concept is exemplified by but not limited to localized contrast agents, fiducial markers visible on the ultrasound image (irrespective of the superimposing of anatomical features). For example, localized injection of an ultrasound contrast agent to a target location in-vivo may allow imaging of the target via ultrasound, and locating the tracked particle in relation to the target. See Kun Zhang, et al. “Marriage Strategy of Structure and Composition Designs for Intensifying Ultrasound & MR & CT Trimodal Contrast Imaging,” ACS Appl. Mater. Interfaces, 2015; DOI: 10.1021/acsami.5b04999:

In one embodiment, the fiducial markers are used to follow the bulk motion of the organ/patient, thereby tracking the microbot position relative to the shifted organ, where without this reference, a large bulk shift in organ position (e.g. due to breathing), much larger than microbot translational movement might result in wrong estimate of microbot location relative to patient.

### Signal Processing

According to some embodiments, in a system utilizing N>1 ultrasound sensors located at different locations around the operation volume, it is possible to increase SNR and reduce the noise by averaging the greyscale signal at any given location across various US (Ultrasound) sensors, using a variety of techniques commonly used for 3D ultrasound imaging (USCT). In a simplified example, assuming the noise captured at a given pixel by a given sensor is independent of the noise at other sensors, averaging of greyscale image value across N sensors reduces the noise by a factor of N\hat{ }(½). In an ideal scenario, It is known exactly how to map the image of one US sensor to that of another (i.e., mapping from x1, y1 pixel coordinate in image 1 captured by sensor 1, to x2, y2 pixel in image 2 captured by sensor 2). In practice, this mapping f(x1,y1)=(x2,y2) may be noisy due to different characteristics/echoes of the imaged medium, different orientations of the sensors, etc. For example, it is possible that the mapping f would be from (x1,y1), to (x2, y2)±k pixels. Hence, this averaging procedure would require pre-calibration to estimate the various noise parameters (such as k in the example above). To conduct such a calibration procedure, several fiducial markers located externally on the operation volume can be used to calibrate sensor location and SNR estimate with respect to each other in 3D. Such fiducial markers can be distinct high-contrast shapes placed in specific 3D locations. Each US sensor's location and orientation in 3D can be calculated analytically by comparing the location of said fiducial markers on the image captured by said sensor (triangulation). Denote the resulting empirically derived mapping function as F(x1,y1). At the same time, the 3D location and orientation of each US sensor can be accurately defined via external means (e.g., via a separate calibrated 3D sensor or camera taking a snapshot of US sensor locations). This allows accurate calculation of the theoretical mapping f(x1,y1) (absent any noise). Now, it is known that F(x1,y1)=f(x1,y1)+σ (where σ is a noise factor). Evaluating F and f in various fiducial marker locations allows accurate estimate of the noise factor and calibrating the noise reduction mechanism (such as the averaging mechanism described above). For example, if σ=±k pixels, one may choose to average the images across the various sensors at different image offsets up to k pixels, and estimate the location of the particle at a given point by comparing the averaged image SNR across all the offsets. If a particle is present in a given image window, the averaged SNR can stand out at one of the offsets. If a particle is not located, then the SNR can remain low regardless of offset. Note that the logic above applies per pixel or per any larger bin which could be used as a minimal unit for particle tracking, consisting of multiple pixels.

In one embodiment, instead of using multiple sensors it is possible to use a single sensor with a combination of multiple physical masks distorting the ultrasound waveform and image (see example in Pieter Kruizinga, et. al. “Compressive 3D ultrasound imaging using a single sensor,” Sci. Adv. 2017; 3: e1701423.) One can then use a noise averaging procedure as described above, or another signal processing algorithm, such as the one described in Kruizinga et. al., utilizing the data received from the multiple image variations collected by a single sensor (instead of multiple sensors). This would allow SNR increase and better localization of the particle in the image. The innovative step in this case is derived from the use of the noise averaging technique/signal processing for binary classification of particle presence per pixel or per image bin (yes/no) and more accurate identification of particle location, rather than the generation of an accurate grayscale image.

For example, let us assume that the sampled grayscale value of a pixel is defined as X=E(X)+N(0,σ), with the baseline noise standard deviation σ, and the average sampled value is m. For N1 samples, it is 95% confident that (abs(E(x)−m))<1.96*σ/sqrt(N1), using 2-sided Z score. Assume one is interested in being 95% confident that (E(X)−m)<err (i.e, the difference between the average sampled value and the real pixel value, is below a given fixed threshold). It is hence required then to take N1 averaged samples such that 1.96*σ/sqrt(N1)<err. So:

N1>(1.96*σ/err)\hat{ }2 Equation 1:

Now, assuming that instead one wants to solve the classification test, i.e., show that E(x)>thr, where thr=fixed signal threshold distinguishing tissue from the object one wants to identify. Let us assume one wants to be 95% confident this is the case. Hence, one needs to choose N2 samples such that (m>thr+1.65*σ/sqrt(N2)), using one-sided Z score. So,

N2>(1.65*σ/(m−thr))\hat{ }2 Equation 2:

According to some embodiments, in a real world application, it is required that m-thr>=err (i.e., the gap required for classification above the threshold is greater or equal than the allowed error for a grayscale image) In other words, if you are not sure enough of a pixel's grayscale value in relation to the threshold (using the naked eye), you can not be sure you are above the threshold. So,

(1.96*σ/err)\hat{ }2>(1.65*σ/(err))\hat{ }2>(1.65*σ/(m−thr))\hat{ }2

Hence, any N1 satisfying Equation 1, by definition satisfies Equation 2.

In other words, Equation 1 is a stronger requirement in terms of sample size than Equation 2 (i.e., one can choose N2 which can satisfy Equation 2 but not Equation 1). Hence, the classification problem is an easier problem to solve (in terms of SNR maximization) than the SNR maximization needed for accurate grayscale evaluation.

In one embodiment, a reference image is taken with particle in a stationary position X, where the US scan head is held with a robotic arm to maintain the exact position of the scan head relative to the imaged region. After a particle progresses, the reference image is subtracted from following images, thereby isolating the regions in the image caused by particle shift only (hence—particle location). Post processing algorithm to detect particle in the subtracted image can search for particle location only in the region where particle could have progressed to during the time of propelling, knowing its maximal possible average velocity.

In another embodiment, it is possible to utilize the raw ultrasound signal as received by the piezoelectric element prior to any additional hardware signal processing. Assuming that the particle is made of a rigid material with greater reflective/scattering properties than the surrounding soft tissue, it is expected that the received signal would be expected to be stronger for high frequency components. Recall that the goal is not to identify fine features in the tissue (lower frequency components) but a binary classification of data per image bin, indicating the presence of a particle. Note that at the raw signal stage it is referring to high frequency components in time, not in space. Hence, applying a high pass filter on the raw data would allow better isolating the relevant information content of the data stream (related to presence of particle) at the expense of unnecessary information content (tissue gradations) Similarly, a high pass filter or another similar image processing mask can be applied to the post-processed B-mode image. Note that now it is talking about a high frequency component in space (i.e., sharp changes in the image across adjacent image bins, indicating the presence of a particle).

In another embodiment utilizing several ultrasound sensors, a single ultrasound transducer signal can generate input for other sensors (as well as for the sensor associated with the original transducer). The combination of such inputs can then be analyzed in an integrated manner, further increasing SNR and improving the ability to identify the location of particle in tissue. In a specific example, let us assume two sensors are positioned in diametrically opposing positions, with the particle located in the middle between them, inside tissue. The wave from sensor 1 is reflected by the particle, returning to sensor A as a strong, high amplitude echo. This signal does not reach sensor 2, as it is entirely reflected by the particle (in the ideal scenario). As one shifts sensor 1 away from the particle location, it no longer receives a strong echo (as the signal travels through tissue and is not reflected). This signal travels all the way to sensor 2, where it is received. The combination of the two signals received by the sensor allows meaningful increase in the SNR, as it is significantly increases the information content of the signal (i.e., one no longer relays only on the reflected signal towards sensor 1, but also on the signal component traveling through tissue to sensor 2).

### MEMS Based Ultrasound Signaling

According to some embodiments, it is possible to incorporate inside the particle a MEMS component (integrated circuit, along with an energy source) allowing active vibration of a cantilever/a membrane/another micro-mechanical component utilizing a piezoelectric element, in a particular mechanical frequency. The energy to power the integrated circuit (IC) is derived internally from a battery or another localized energy source, or transferred to the particle remotely, through other system components.

According to some embodiments, another option is where said cantilever/membrane/micro-mechanical component is vibrating at a given resonant frequency in response to external stimuli, such as the application of an external magnetic field on a magnetic elastomer membrane, or a focused ultrasound beam at a particular frequency band.

According to some embodiments, the vibration of the cantilever/membrane can enhance the visibility of the particle for the ultrasound imaging system.

### Specific Particle Geometry

According to some embodiments, a particular particle geometry may be used to enhance the reflection/scattering pattern of the particle, or to generate a particular pattern on the image and improve tracing of the particle. In one embodiment, a particle may be designed to have a particular shape which facilitates identification via an image processing algorithm, such as convolution or cross-correlation with a predefined mask corresponding to the known shape. Of particular use are shapes with spherical symmetry (for the particle or for a component of the particle), allowing identification irrespective of sensor positioning in relation to the particle. In another embodiment, the particle geometry can facilitate the use of a particular signal processing filter applied on the image (e.g., the inclusion of periodic grooves on particle surface which can be identified using autocorrelation of the image or a band pass filter corresponding to groove periodicity). See U.S. Pat. No. 4,401,124 (Guess et al.).

### Specific Particle Coating

In one embodiment, therapeutic particles may exhibit a specific surface coating and/or multi-layered composition to facilitate image enhancement. Examples of such particle design can be found in: Kun Zhang, et. al. “Double-scattering/reflection in a Single Nanoparticle for Intensified Ultrasound Imaging,” Sci. Rep. 2015, 10.1038/srep08766; Jun Chen, et al., “Theranostic Multilayer Capsules for Ultrasound Imaging and Guided Drug Delivery,” ACS Nano 2017, 11, 3135-3146: Dennis Manuel Vriezema, et. al. “Coating for Improving the Ultrasound Visibility”; and US 2014/0207000 A1 (Vriezema et al.).

### Specific Chemical Agent/Contrast Agent Inside Particle (e.g., Microbubbles)

In one embodiment, therapeutic particles may exhibit specific internal compartments designed to enhance imaging as exemplified but not limited to micro-nanocavities, micro-nanorattles, micro-nanoinclusions or other micro-nanoirregularities, as exemplified in FIG. 2.

In one embodiment, therapeutic particles may exhibit specific image enhancing inclusions, cavities, containers that release agents or modalities that enhance imaging as exemplified but not limited to contrasting agents or microbubbles. Examples of such particles are described in Shu-Guang Zheng, et al. “Nano/microparticles and ultrasound contrast agents,” World J Radiol 2013 Dec. 28; 5(12): 468-471 and María-Victoria Alvarez-Sánchez and Bertrand Napoléon, World J Gastroenterol. 2014 Nov. 14; 20(42): 15549-15563.

In one embodiment, therapeutic particles may feature particular chemical and/or biochemical molecules, as exemplified by but not limited to Janus alloys, (micro) electrodes, Pd/Pd-alloys, specific redox or metabolic enzymes that facilitate local production of externally traceable substances that include gases and/or specific detectable metabolites.

### Tissue-Specific Particle Design to Enhance Imaging

In one embodiment, a surface of particle is modified with iodine-containing agents via absorption, complexation, covalent modification or incorporation as exemplified but not limited to a polymer film (ex., PLGA) that contains iodinated species. The resulting particles are expected to be visualized using CT technology, specifically low tube voltage (80 kV) since the mean photon energy of 47 keV to 56 keV is close to the absorption maximum of iodine (33.2 keV). Therefore, significantly higher levels of contrast can be achieved when using iodine-containing contrast agent at a low tube voltage than at a high tube voltage as described in Pregler, B, et al. “Low Tube Voltage Liver MDCT with Sinogram-Afrmed Iterative Reconstructions for the Detection of Hepatocellular Carcinoma,” Sci. Rep. 2017; DOI:10.1038/s41598-017-10095-6. Specifically, an iodine-containing polymer coating that contains iodinated structural macromolecules or iodinated compounds embedded into coating is used. Similarly, alternative contrasting entities could be incorporated into the particle's structure or coating to enhance SNR. In an additional illustration, a Hf-containing molecule is incorporated into the particle structure via techniques described above to enhance CT-mediated visualization as described in Frenzel, T. “Characterization of a Novel Hafnium-Based X-Ray Contrasting Agent,” Invest. Radiol. 2016, 51(12), 776-785. Yet another embodiment involves localized release of nanoparticles endowed with specific environmental markers that are preloaded onto the aforementioned microparticle. For example, preloaded nanoparticles could be equipped with specific antigens that recognize disease-affected cells or tissue as illustrated by galactose-grafted micelles or liposomes, Yan, G., et al. “Stepwise targeted drug delivery to liver cancer cells for enhanced therapeutic efficacy by galactose-grafted, ultra-pH-sensitive micelles,” Acta Biomater. 2017, 15(51), 363-373.

### Agents to Increase Contrast by Reducing Tissue Signal Noise

In one embodiment, therapeutic particles may exhibit specific image enhancing inclusions, cavities, containers that release agents or modalities that reduce background signal thus enhancing therapeutic particle signal.

### Signal Processing Techniques to Increase Contrast by Reducing Tissue Signal Noise

In another embodiment, a specific raw data processing algorithm is applied in order to reduce or eliminate artefactual interference or background from the tissue as exemplified but not limited to temporal averaging homomorphic Wiener filtering, temporal averaging, median filtering, adaptive speckle reduction, wavelet thresholding, adaptive filtering, anisotropic diffusion or nonlinear diffusion tensor derived from the so-called structure tensors in the Benzarti F., et. al. “Speckle Noise Reduction in Medical Ultrasound Imaging,” Int. J. Comp. Sci. Images 2012; 9(2), 187-194.

According to some embodiments, the goal of a typical tissue signal noise algorithm is to increase SNR for accurate grayscale image formation. In contrast, the goal of the algorithm here is to classify pixels in a binary fashion (particle present or not). Utilizing the nomenclature of Equation 1, 2 above, while a typical tissue signal noise reduction code seeks to minimize o′ (i.e., the noise generated by tissue), one can utilize the algorithm to effectively subtract the background level of tissue signal (lowering thr).

### Specific Particle Motion to Enhance Imaging

According to some embodiments, a particular particle motion may be used to enhance the reflection/scattering pattern of the particle, or to generate a particular pattern on the image and improve tracing of the particle. In one embodiment, therapeutic particles may exhibit specific dynamics and/or motion behavior to facilitate imaging as exemplified by off-gradient axis rocking, rotation, vibration, etc. In one embodiment, a particle may be designed to rotate at a given frequency, generating a particular time-variant reflection/scattering pattern visible using regular ultrasound or Doppler ultrasound. In another embodiment, the combination of particle geometry and predefined particle motion can facilitate the use of a particular signal processing filter applied on the image sequence. In a specific example, inclusion of periodic grooves on the particle surface along with particle rotation, which can generate a period pattern both in time and in space (across the image). This facilitates particle identification, using autocorrelation of the image in time or in x-y-z space, or by applying a band pass filter corresponding to groove periodicity and/or rotation frequency

Interface with Other Modules

In one embodiment, the aforementioned imaging platform or a combination thereof can be used in conjunction with other platforms including external propulsion devices, specific therapeutic particle(s) of particular design and therapeutic modality, specialized delivery and retraction platform and integrating hard-/software.

In one embodiment, the aforementioned imaging platform includes an ultrasound device with operational frequencies of 0.25-50 MHz, electromagnetic or permanent magnet-mediated propulsion platform, (ferro-/para-)magnetic therapeutic particles, particles made of respective composites or materials that feature embedded (ferro-/para-)magnetic materials or microelectromechanical systems (MEMS).

Imaging System with a Non-Ultrasound Imaging Component for the Tracking of the Particles (Standalone, or Hybrid in Combination with Ultrasound)

### Location Detection Using Localized Magnetic Field Gradients

In one embodiment, magnetic field gradients can be used in combination with MEMS for particle detection. In this case the particle is effectively a microbot, with MEMS components.

According to some embodiments, detection of microbot position on each of the three axes (x,y,z) is done separately, each axis at a time.

According to some embodiments, without loss of generality, let's assume applying a fixed (non-rotating) magnetic field gradient Gx along x. The magnetic field in each position on X: Bx=X*Gx. Retrieving from the MEMS particle the magnetic field Bx can uniquely give X. The value of Bx can then be communicated from the particle to an externally located communication receiver connected to the tracking module, which can use this value to deduce the particle location.

According to some embodiments, possible technologies for localized magnetic field sensing by the particle are: a miniature Hall sensor, or magnetically activated semiconductors. Uplink communication capabilities from the particle can be implemented using MEMS based communication modules, such as RF communication, optical, ultrasound, or other communication methods.

### Particle Tracking Using Magnetic Particle Imaging (MPI) as the Tracking Module

MPI is an imaging modality. According to some embodiments, hardware for MPI includes coils to produce magnetic fields. In one embodiment, these may be the same coils that are already exist in the system for the microbot propulsion. The MPI images can be acquired in between propulsions, with instantaneous “imaging episodes”. Specifically, pulses are to exhibit distinct power, frequency and time-resolved profiles as exemplified but not limited to rectangular, double exponential and damped sinewave pulses or a combination thereof that are differentially selected, calibrated and timed to differentiate propulsion and imaging events. In another embodiment, the MPI hardware is separate from the system controlling microbot motion. Specifically, a microbot can carry another cavity/attached capsule, filled with superparamagnetic iron oxide nanoparticles (SPIONs). Particle size of SPIONs is few to tens of nm, so enough particles can be loaded into one microbot. With MPI, no signal can emerge from the tissue, but only from the SPIONs, giving an accurate detection of the microbot, with no background signal.

In one embodiment, the SPIONs can not be contained in a chamber, but can be present by a means of coating of the microbot (SPION-based coating).

In one embodiment, detection of each microbot (out of a fleet of microbot) separately is by providing each microbot with a different concentration, or a different type of SPIONs. This can enable distinguishing between multiple microbots.

According to some embodiments, both ultrasound and optical approaches may be used to reliably and reproducibly identify microbots and microbot dynamics in vitro, ex-vivo and in-vivo. An overview of two experimental protocols (euthanized murine models and anesthetized in-vivo models) is provided below.

In-Vivo Preparation: Microbot Insertion into Euthanized or Anesthetized Animal Liver

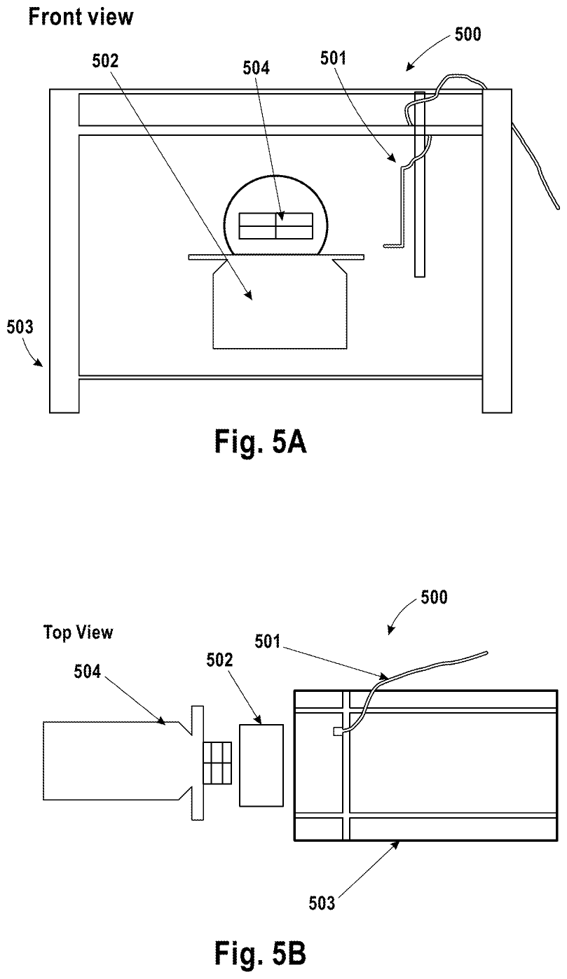

6-10 weeks old Sprague-Dawley (SD) rats were aestheticized using 5% isoflurane in 100% O₂ with sedation to be confirmed with a toe pinch. The anesthesia was maintained at 1-2% isoflurane by inhalation and ventilation throughout the procedure. Following anesthesia induction, a midline incision was made in the skin of the abdomen and a second incision was made into the peritoneal cavity using scissors. A microbot particle (301) was inserted completely into either Right Medial Lobe or Left Lateral Lobe of the rat's (302) liver (303) using plastic forceps (304), as demonstrated in FIG. 3. Needle (20G, ca. 0.91 mm outer diameter) puncture was used as a positive control to assess the liver damage. The puncture was performed via the open-wound procedure to emulate the particle insertion sequence or in-situ through skin.

### In-vivo Setup and Mobility Procedure

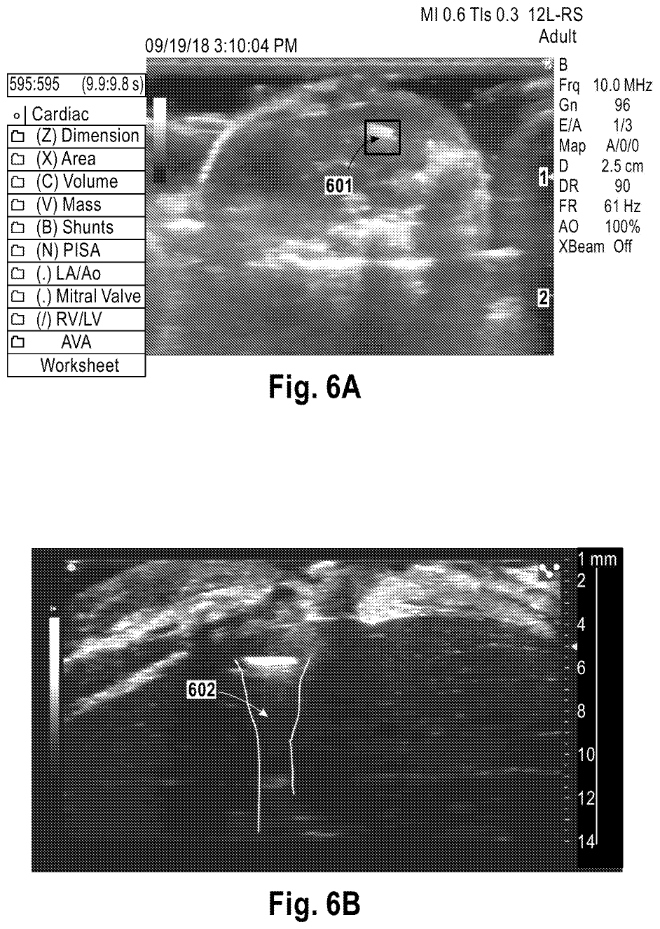

According to some embodiments, an euthanized or anesthetized rat (402) is placed on a stage (403) for imaging either laterally or horizontally with respect to the Macho 2.5 magnet array (401) as demonstrated in FIG. 4.

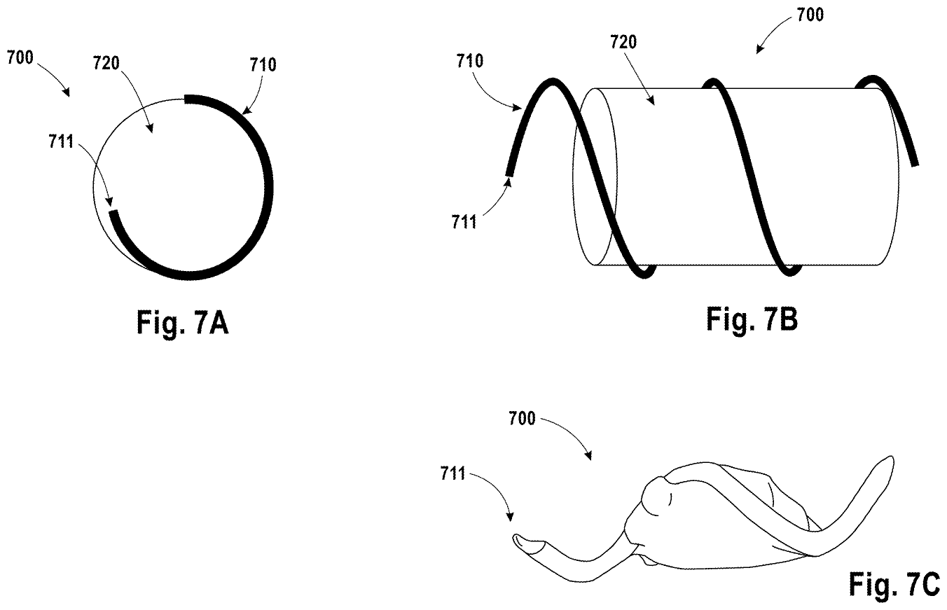

According to some embodiments and as demonstrated in FIGS. 5A and 5B (Front and top views), to further facilitate ex-vivo and in-vivo operations, a specific device or setup (500) is disclosed, configured for the controlled positioning and movement of the ultrasound (“US”) probe (501). The specific setup is configured to allow the head of a modified 3-dimentional printer (503) to accommodate the US probe (501) and to move independently of the stage (502) in the z,y axis with the base stationary and the stage allowed to move in three dimensions. FIG. 5 demonstrates a commercially available ADIMLab 3D Printer Assembly 24V Prusa I3, an “off the shelf” 3D printer (503) that encompasses two axes that move as one unit, with the third moving independently. The modification is introduced to amend the mobility about the z,y axis and to fuse them to the x axis stage. The resulting system moves as one unit with dynamics achieved in three dimensions. Considering the need to work in a magnetic environment (e.g. magnet array 504), further modification of the ultrasound probe-printer array entailed fabricating a non-magnetic 3D printer head fixture of a non-magnetic material, in this instance aluminum.

### Ultrasound-Based Imaging of Microparticles Ex-Vivo and In-Vivo

According to some embodiments, once the rat is positioned on the stage, ultrasound gel is placed on the abdomen of the rat. The ultrasound probe is lowered onto the rat orthogonal or parallel to the rat abdomen. The GE Logic E machine is subsequently tuned to an operational frequency of 5 to 50 MHz and set to a preinstalled configuration for viewing the abdomen or carotid arteries.

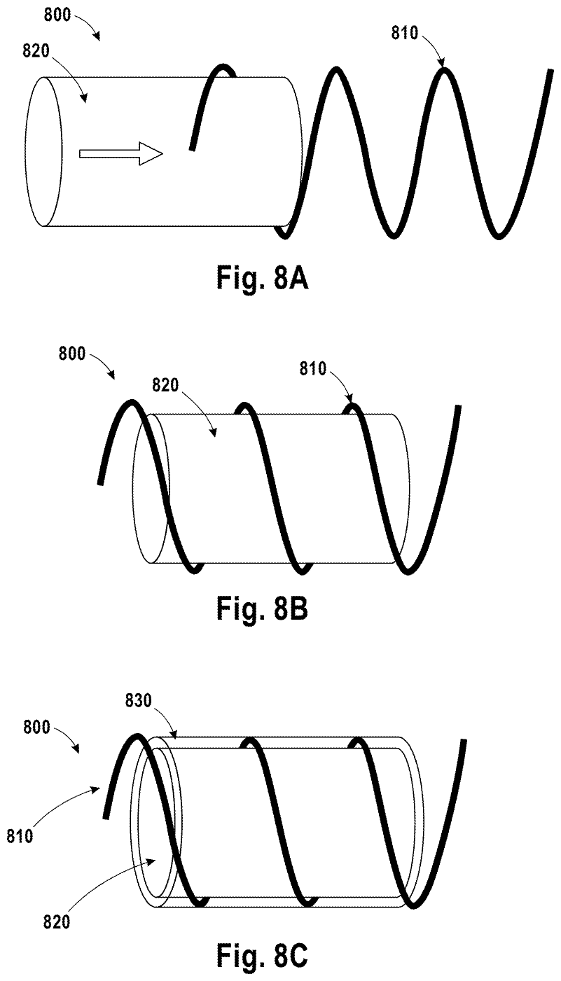

According to some embodiments, in a standard imaging protocol, the generated ultrasound beam is moved in a scanning fashion across the abdominal cavity until the microbot is found. According to some embodiments, depending on the dimensions and topology of the microbot, the particle (601) can be reliably identified by looking for outlines on the ultrasound wave reflection image, as demonstrated in FIG. 6A. According to some embodiments, during image adjustment, the gain can be modulated further to maximize the difference between the background and the microbot to yield a better contrast between the surrounding tissue and a particle.

According to some embodiments, if topology and size of microbot are comparable to that of the artifacts in the liver affecting visibility, slight rotation of a magnet to alter the microbot's positioning is sufficient to recapture the image via ultrasound.

According to some embodiments, recording of the ultrasound footage is mediated via an AV.io HD-Grab and Go USB video capture software connected to a Windows machine. Post-imaging analysis involved tracking of the microbot on the screen to identify both its position and velocity. The tracking software takes a frame-by-frame comparison of the ultrasound video that is analyzed pixel by pixel using color schemes in Python via OpenCV. The motion of a microbot is ascribed to a predefined significant difference with subsequent frame vs the previous one at certain pixels and past a predetermined motion threshold.

In addition, according to embodiments of the invention and as demonstrated in FIG. 6B, a spring-magnet based combination particle (602) can be visualized using a ‘lighthouse beam’ configured ultrasound. Under these irradiation conditions, the beam dynamics appear to follow the microbot rotational movement. Specifically, ultrasound reflection from the magnet appears as a very distinct uneven line under the actual microparticle.

According to some embodiments, an alternative way to improve an ultrasound-based imaging is to incorporate a capacitive micromachined ultrasonic transducer (CMUT) to the microbot. The energy transduction in CMUT is due to change in capacitance. In the proposed design, a CMUT, a microcapacitor or a similar circuit that incorporates an ‘asymmetric’ magnetic surface (partially magnetic, uneven magnetic coating or like) is a part of the microbot. The resulting microdevice is expected to change capacitor's volume when treated with a magnetic field of a distinct frequency range (kHz-MHz) vs rotational magnetic frequency (Hz). This step is to yield a distinct detectable acoustic signal detectable externally and unequivocally associated with the particle.

### Ex-Vivo Imaging of Microbots Using Optical Upconversion Phosphors Approach

According to some embodiments, an optical upconversion-based protocol is further introduced for imaging in order to supplement visualization of the microparticles in vitro, ex-vivo and in-vivo.

### Upconversion-Based Imaging

According to some embodiments, Upconversion Phosphor (UCP)-Embedded Microbots and Upconversion Imaging System were utilized for In Situ Tracking of Microbots Ex-vivo. Rare-earth ion doped upconversion phosphors (UCPs) absorb two or more near-infrared photons (usually 800 nm or 980 nm) and upconvert them to higher energy photons. Wavelengths of the upconverted photon depend on dopant types and their doping densities. Here, demonstrated are upconversion imaging of sodium yttrium fluoride (NaYF₄) microcrystals (particle sizes 1-5 μm) doped with trivalent ytterbium (20%) and erbium ions (3%). These doping densities absorb 980 nm photons and upconvert them into green and red photons. Because there is less absorption and scattering of 980 nm photons in tissue than visible photons, it allows for the excitation light to reach deeper into tissue and thus allows one to image deeper through tissue. Furthermore, because UCPs use NIR excitation, a wavelength at which there is minimal autofluorescence, the signal-to-noise ratio of the upconverted luminescence is large. This allows for a high-contrast, deep tissue detection of microbots in situ.

### Upconverting Microbot Fabrication

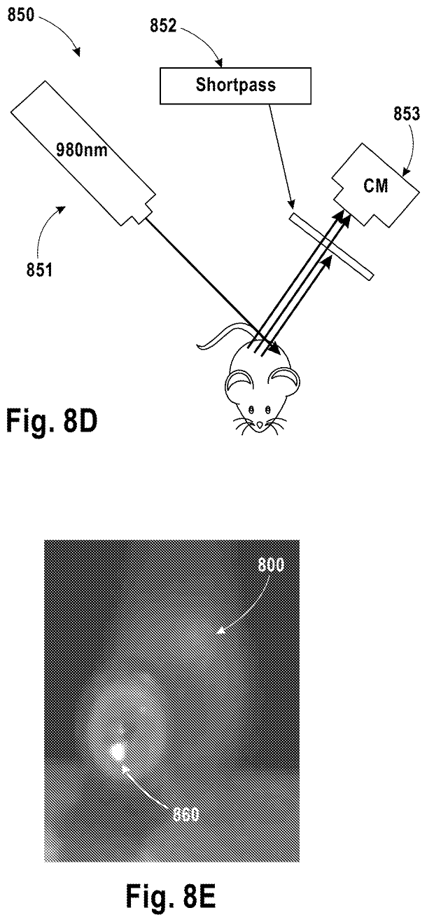

According to some embodiments, and as demonstrated in FIGS. 7A, 7B and 7C, the microbot (700) comprises a piece of stainless-steel compression spring (710) (e.g. wire diameter: 0.152 mm; inner diameter: 0.610 mm; coil periodicity ˜0.4 mm) is axially extended to have a coil pitch between 0.7 mm and 1.5 mm. The extended spring is then clipped off with nipper pliers to provide a sharp, chiseled tip (711), as it can be seen in FIG. 7C. This sharp tip allows for the microbot to easily pierce through matrix. According to some embodiments, at a position between 0.2 mm to 1.3 mm from the tip, a radially magnetized, cylindrical nickel-plated neodymium (N52) magnet (720) (diameter of 0.5 mm and length of 1 mm) is axially aligned. Then carefully, a layer of sodium yttrium fluoride micro-particles doped with rare-earth ions (e.g. Er³⁺, Yb³⁺, Tm³⁺) is dusted onto the magnet. Then a droplet of cyanoacrylate is deposited on top of the microbot to (1) adhere the micro-particles on the magnet and (2) fix the position of the magnet on the spring. The entire microbot is gently dabbed with paper towel to remove excess adhesive. Then, the process of dusting the microbot with sodium yttrium fluoride micro-particles and applying cyanoacrylate is repeated twice more. Subsequently, two additional layers of cyanoacrylate is applied to affix any remaining components. After the application of cyanoacrylate, the microbot is dried in air overnight. The following day, the other end of the spring is clipped off with a nipper plier at a distance between 0.2 mm to 1.3 mm from the magnet. The longitudinal view of the microbot (700) after construction is depicted in FIG. 7C.

### Optical Illumination and Collection Setup for Upconversion Imaging

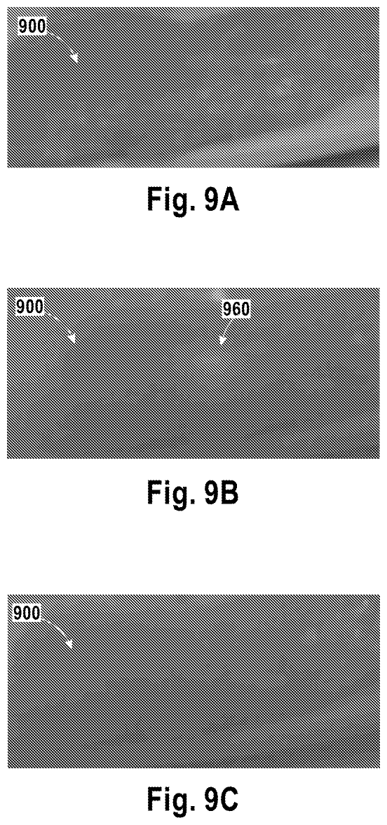

FIGS. 8A, 8B and 8C depict a fabrication of a microbot (800), comprising a cylindrical magnet (820) aligned within a spring (810), and coated (830) with UCPs (e.g. dusted with upconversion phosphor and glued with cyanoacrylate), according to some embodiments of the invention. As demonstrated in FIG. 8A, first the magnet (820) is inserted into the spiral spring (810); then as demonstrated in FIG. 8B, the magnet's longitudinal axis is aligned with the springs longitudinal axis, and then as demonstrated in FIG. 8C the microbot (800) is coated with UCPs (830).

According to some embodiments, the UCPs present on the microbot absorb incident 980 nm excitation, which upconverts it into visible luminescence. UCPs doped with trivalent ytterbium and erbium emit green and red luminescence.

An optical setup (850) for upconversion imaging is provided in FIG. 8D, according to some embodiments of the invention. A 980 nm laser source (851) (1 Watt diode-pumped solid-state laser; Civilaser) is positioned to irradiate UCP-coated microbot embedded in tissue. The 980 nm laser penetrates through up to about 10 mm of tissue and excites UCP-coated microbots (not shown, within the rat). The upconverted green and red luminescence gets collected through a shortpass filter (852) (Schott KG-5) that transmits green and red visible fluorescence, while rejecting 980 nm excitation source. Finally, a CMOS camera (853) (e.g. AmScope MU5 with a 35 mm C-mount lens) is placed after the filter to image the upconverted luminescence. FIG. 8E shows green fluorescence (860) from trivalent ytterbium and erbium doped UCPs upon 980 nm excitation.

### Resulting Illumination

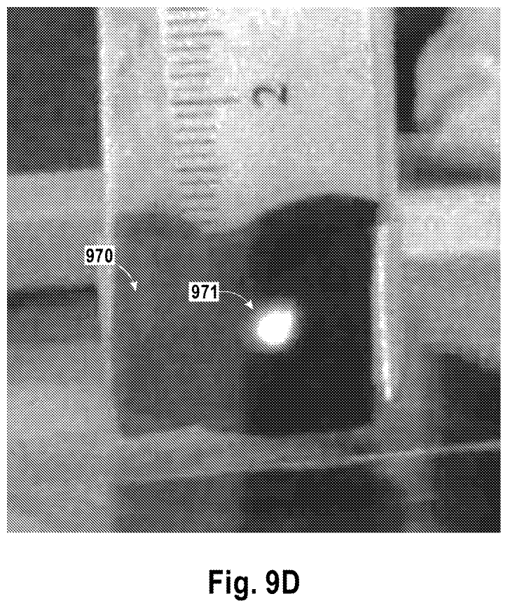

According to some embodiments, to demonstrate upconversion imaging capabilities ex-vivo, a UCP-embedded microbot is placed underneath a 5 mm thick sample of pork (900) as shown in FIGS. 9A, 9B and 9C. FIG. 9A demonstrate that under conventional imaging, one cannot visualize the UCP-bot that is underneath the pork. Upon excitation with 980 nm laser, the incident irradiation penetrates through at least 10 mm of pork and excites the UCPs. Subsequently, the UCPs produce green and red upconverted luminescence, which scatter back through tissue, as it is shown in FIG. 9B. While one would expect red fluorescence to be more dominant, as there is greater scattering through tissue in green wavelengths (960) than in red, because the dopant density is optimized to emit higher intensities of green than red, the upconverted images show a green scattered spot. When the UCP-bot is removed from underneath the pork (with 980 nm illumination still pointed at the same position) the green luminescence disappeared as demonstrated in FIG. 9C. This provides evidence that the green luminescence originates solely from UCPs present on the microbot surface.

According to some embodiments, when the UCP-embedded microbots are embedded in a different medium, such as for example a turkey liver (970), they exhibit a slightly different upconversion imaging properties. Due to the nature of turkey liver, which absorbs more green light than pork loin, the emitted green to red ratio varies in it. As an example, the orange luminescence (971) visible in FIG. 9D is also an upconverted luminescence from an identical UCP-embedded microbot as in FIG. 9B. FIG. 9D exhibits a strong orange luminescence, which indicates that more green luminescence is absorbed by turkey liver than in pork, thus resulting in a more orange-colored luminescence.

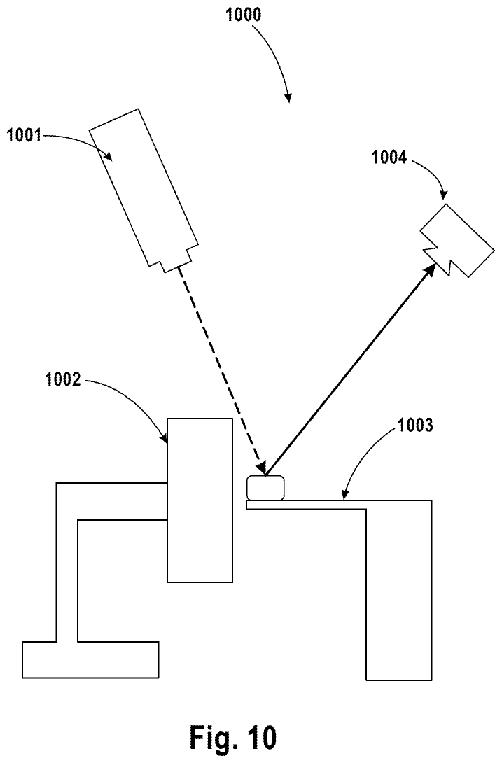

FIG. 10 demonstrates the setup (1000) used for ex-vivo illumination, according to some embodiments of the invention, comprising: a 980 nm laser source (1001), a Macho 2.5 magnet (1002), an ex-vivo liver platform (1003) and the CMOS detector (1004).

In summary, the foregoing demonstrated: fabrication of UCP-embedded microbots, and successful setup and demonstration of upconversion imaging ex-vivo. In both 5 mm thick pork loin as well as 2.5 mm thick turkey liver, the Examples exhibit suitably bright upconversion images that can allow movement of microbots in situ. The Yb³⁺ and Er³⁺ doped NaYF₄ crystals showed a range of green to orange luminescence depending on absorption properties of tissues.

The specific examples herein are illustrative of the invention as recited in the appended claims, and are not to be deemed as limiting the invention. Likewise, inventions are disclosed herein which are not yet reduced to claims. Rights to claim disclosed but unclaimed subject matter are reserved.

While, certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An imaging system for tracking nano- or micro-particles, the system comprising:
an ultrasound imager having plurality of ultrasound sensors driven by a single ultrasound transducer signal, the imager configured to sample at a sampling rate in the kHz-MHz range;
a plurality of particles having an image enhancement feature facilitating detection in a patient or an in-vivo environment, the particles having a size in a micrometer or nanometer range; and
a display configured to display the particles in the patient or in-vivo environment via the ultrasound imager.

2. The imaging system of claim 1, further comprising a low voltage CAT scan (CT) technology configured to display the particles in the patient or in-vivo.

3. The imaging system of claim 1, wherein the ultrasound imager is operative at a processing delay of one second or more.

4. The imaging system of claim 1, wherein the ultrasound imager is operative in accordance with a standard operating or optimized procedure providing multi-organ resolution of up to 50 microns.

5. The imaging system of claim 1, wherein the ultrasound imager is configured to process feedback signals through a specialized standard or custom algorithm so as to enhance signal-to-noise ratio (SNR).

6. The imaging system of claim 1, wherein the image enhancement feature is implemented as a coating containing iodine.

7. The imaging system of claim 1, wherein the image enhancement feature is implemented as a surface irregularity.

8. The imaging system of claim 1, wherein the image enhancement feature is implemented as a result of particle dynamics or specific motion frequency, as exemplified by Doppler effect.

9. The imaging system of claim 2, wherein the low voltage CT technology is operative at 50-300 kVolt.

10. The imaging system of claim 1, further comprising a magnetic imaging system configured to track the particles.

11. The imaging system of claim 10, further comprising a propulsion system configured to advance the particles through the patient or in-vivo environment via a series of magnetic propulsions.

12. The imaging system of claim 11, wherein the magnetic imaging system is configured to capture position images of the particles in the patient or in-vivo environment in between the magnetic propulsions.

13. The imaging system of claim 11, wherein the image enhancement feature is implemented as a load of a superparamagnetic iron oxide nanoparticles (SPION) and/or mesoporous silica nanoparticles (MSN).

14. A magnetic imaging system for tracking conveyable, therapeutic nano- or micro-particles, the system comprising:
a magnetic imager configured to track the particles in a patient or an in-vivo environment;
a plurality of conveyable, therapeutic nano- or micro-particles loaded with superparamagnetic iron oxide nanoparticles (SPION) or mesoporous silica nanoparticles (MSN); and
a display configured to display the particles in the patient or in-vivo environment, via the magnetic imager.

15. The magnetic imaging system of claim 14, further compromising a low voltage CAT scan (CT) technology configured to display the particles in the patient or in-vivo environment.

16. The magnetic imaging system of claim 15, wherein the low voltage CT technology is operative at 80 kVolt.

17. The magnetic imaging system of claim 14, further comprising a propulsion system configured to advance the particles through the patient or in-vivo environment through a series of magnetic propulsions.

18. The magnetic imaging system of claim 17, wherein the magnetic imager is configured to capture position images of the in the patient or in-vivo environment in between the magnetic propulsions.

19. The magnetic imaging system of claim 14, further comprising an ultrasound imager having a plurality of ultrasound sensors driven by a single ultrasound transducer signal.

20. The magnetic imaging system of claim 19, wherein the conveyable, therapeutic particles have an iodine coating.

21. A method for tracking conveyable, therapeutic nano- or micro-particles in a patient or an in-vivo environment; the method comprising:
sampling the patient or the in-vivo environment at an ultrasound sampling frequency in kHz-MHz range with intermittent gaps of one or more seconds between subsequent ultrasound applications;
processing sample feedback of the conveyable, therapeutic particles in the patient or the in-vivo environment with a protocol providing multi-organ resolution up to 50 microns; and
displaying the objects on a display.

22. The method of claim 21, further comprising: employing low voltage CAT scan (CT) technology and displaying the CT scanned objects on a display.

23. The method of claim 22, wherein the low voltage CT technology is operative at 80 kVolt.

24. The method of claim 21, further comprising propelling the conveyable, therapeutic particles in the patient or in-vivo environment through magnetic propulsions.

25. The method of claim 24, further comprising magnetically imaging the conveyable, therapeutic particles in the patient or in-vivo environment in between the magnetic propulsions.

26. An imaging system for tracking nano- or micro-particles, the system comprising:
nano- or micro-particles having embedded rare earth ion-doped phosphors;
an upconversion energy source configured to provide energy sufficient to upconvert photons of the ion-doped phosphors to a visible range;
a detector having plurality of sensors configured to detect luminescence from the nano- or micro-particles; and
a display configured to display the particles in the patient or in-vivo environment based on the detected luminescence.

27. The imaging system according to claim 26, wherein the detector is a system comprising a Complementary Metal Oxide Semiconductor (CMOS) detector and a shortpass filter positioned between the luminescence in the in-vivo environment and the CMOS detector; and wherein the upconversion energy source is a laser configured to provide excitation energy at a wavelength of 800.

28. The imaging system according to claim 27, wherein the rare earth ion-doped phosphors comprise Yb³⁺ and/or Er³⁺ doped NaYF₄ crystals.

29. A method of tracking a nano- or microparticle in an in-vivo environment, comprising:
coating at least one metallic nano- or microparticle with rare earth ion doped upconversion phosphors;
implanting said nano or microparticle coated with rare earth ion doped upconversion phosphors in said in-vivo environment;
exciting the upconversion phosphors to produce luminescence;
imaging the luminescence with a camera; and
detecting the position of the nano- or microparticles in-vivo.

30. The method according to claim 29, wherein exciting the upconversion phosphors is done with a laser configured to provide excitation energy in a range of about 800 nm to about 980 nm, wherein said camera is a complementary metal oxide semiconductor (CMOS) detector, and said luminescence is visible red and/or green light.

31. The method according to claim 29, further including moving the nano- or micro-particle in the in-vivo environment with a magnetic field.

32. A method of making nano- or micro-particles configured to be tracked in an in-vivo environment, comprising:
providing a compression spring;
clipping an end of the compression spring to form a sharp end of the compression spring;
axially aligning a magnet with the compression spring;
positioning rare earth ions onto the magnet; and
adhering the magnet to the compression spring.
